(19) United States
(12) Patent Application Publication
(10) Pub. No.: US 2011/0046482 A1
(43) Pub. Date: Feb. 24, 2011

(54) METHODS FOR REDUCING TOXICITIES ASSOCIATED WITH MEDICAL PROCEDURES EMPLOYING RADIOGRAPHIC CONTRAST AGENTS

(75) Inventors: Edward A. Neuwelt, Portland, OR (US); Leslie L. Muldoon, Tigard, OR (US)

Correspondence Address: SEED INTELLECTUAL PROPERTY LAW GROUP PLLC, 701 FIFTH AVE, SUITE 5400, SEATTLE, WA, 98104, US

(73) Assignee: OREGON HEALTH & SCIENCE UNIVERSITY, Portland, OR (US)

(21) Appl. No.: 12/377,840

(22) PCT Filed: Aug. 16, 2007
(86) PCT No.: PCT/US07/18388

§ 371 Date: Nov. 4, 2010

Related U.S. Application Data
(60) Provisional application No. 60/837,974, filed on Aug. 16, 2006.

(51) Int. Cl. A61M 37/00; A61K 31/195; C07C 321/04; A61P 13/12; A61B 6/00

(52) U.S. Cl. 600/433; 514/562; 562/557; 604/508

(57) ABSTRACT

Improved methods of administration of thiol-based agents, such as NAC (N-acetylcysteine) and STS (sodium thiosulfate), are provided that protect against renal and other organ injury caused by diagnostic or therapeutic intra-arterial procedures which employ radiographic contrast agents.

### FIELD OF THE INVENTION

The present invention relates generally to improved methods for administration of N-acetylcysteine (NAC) and other thiol-based compounds concurrently with, before or after procedures which employ radiographic contrast agents, and provides protective effects to prevent or diminish organ damage, such as renal damage, caused by those agents.

### BACKGROUND OF THE RELATED ART

Radiographic contrast agents are chemicals used in a variety of therapeutic and diagnostic medical procedures in order to enhance images of internal organs and to increase contrast between a target organ and surrounding tissues. However, the use of radiographic contrast agents can lead to acute renal failure even when measures are taken to reduce their toxicity. For example, the nephrotoxic effects of intra-arterial catheterization and infusion of radiographic contrast agents prolong hospital stays, add to the cost of medical care, and can be fatal. The incidence of radiographic contrast agent-induced acute renal failure, currently estimated to be as high as 50 percent among patients with diabetes mellitus and preexisting renal disease who receive contrast agents, is likely to remain high as the use of invasive intra-arterial procedures to diagnose and treat complex disease continues to grow. Prevention or mitigation of renal failure after the administration of a radiographic contrast agent has been notably difficult. Calcium-channel antagonists, adenosine antagonists, and dopamine have all been used without convincing evidence of benefit.

There are several thiol-based chemoprotectant agents that contain a thio, thiol, aminothiol or thioester moiety, several of which have been shown to provide protection against at least some of the systemic toxicities caused by alkylating chemotherapeutics. Illustrative thiol based chemoprotective agents include N-acetylcysteine (NAC), sodium thiosulfate (STS), GSH ethyl ester, D-methionine, and amifostine (also known as Ethyol or WR2721).

NAC is a cysteine analog used clinically to treat respiratory disorders and acetaminophen toxicity (Corcoran et al., J Pharmacol Exp Ther 238:54-61, 1986; McLellan et al., Carcinogenesis 16:2099-2106, 1995). Intravenous (IV) NAC is widely used for acetaminophen overdose and is approved by the FDA for intravenous use in the U.S. The current recommended dose of NAC for acetaminophen overdose is 150 mg/kg infused IV over 15 minutes, with a maintenance dose of 50 mg/kg IV over 4 hours followed by 100 mg/kg over 16 hours. However, the most efficacious route of administration and the length of treatment for this indication remain controversial (Perry et al., J Pediatr 132:149-152, 1998).

Several studies have explored the role of NAC in the prevention of contrast-induced nephropathy (CIN) (e.g., Bagshaw et al., Arch Intern Med 166:161-166, 2006). For example, Pannu et al. performed a systematic review and meta-analysis to assess the efficacy of NAC for preventing this complication (Pannu et al., Kidney International 65:1366-1374, 2004). This study concluded that NAC may reduce the incidence of acutely increased serum creatinine after administration of IV contrast agent, however this finding was of borderline statistical significance, and there was significant clinical heterogeneity between trials. The heterogeneity included different subject populations, different inclusion criteria, different intervention protocols (including NAC dose and route of administration) and different primary outcomes.

Thus, despite considerable interest and numerous clinical studies, consistent and successful strategies for using NAC in the prevention of contrast-induced nephropathy have been elusive. Many studies evaluating NAC in the prevention of contrast-induced nephropathy have used oral NAC, with mixed results. In addition, mixed outcomes have been observed in a smaller number of studies that have evaluated IV infusion of NAC. Doses described in these IV studies include 150 mg/kg 30 minutes prior to and 50 mg/kg 4 hours after contrast agent (Baker et al., J Am Coll Cardiol 41:2114-2118, 2003); 1200 mg 12 hours prior to contrast agent and 1200 mg immediately after contrast agent (Kefer et al., Acta Cardiol 58:555-560, 2003); 500 mg prior to contrast agent (Webb et al., Am Heart J 148:422-429, 2004), and 1000 mg prior to and 1000 mg after contrast agent (Rashid et al., J Vasc Surg 40:1136-1141, 2004).

There remains a significant need in the art for improved compounds and methods that can be used with intra-arterial catheterization and other diagnostic and therapeutic procedures to reduce organ toxicity caused by the use of radiographic contrast agents. Additionally, there is a need in the art for methods for improving dosing and/or delivery strategies for administration of thiol-based radiographic protectants, such as NAC and STS (sodium thiosulfate), and to take better advantage of their pharmacokinetic properties. The present invention fulfills these needs and offers other related advantages.

### SUMMARY OF THE INVENTION

The present invention relates generally to improved methods for administration of N-acetylcysteine (NAC) and other thiol-based agents concurrently with, before or after, administration of radiographic contrast agents, and provides protective affects to prevent or diminish organ damage, particularly renal damage, caused by contrast agents.

According to one aspect of the invention, a method is provided for reducing organ toxicity associated with a medical diagnostic or therapeutic procedure which employs a radiographic contrast agent by intravenously administering at least one dose of a thiol-based agent, such as NAC, in the range of about 600-1500 mg/kg. In certain embodiments, the thiol-based agent is administered intravenously at a dose in the range of about 600-1200 or about 800-1200 or about 800-1500 mg/kg. The thiol-based agent can be administered before, during and/or after administration of radiographic agent. In certain embodiments, the thiol-based agent is administered no more than about 4 hours before or about 8 hours after administering a radiographic contrast agent. In certain other embodiments, the thiol-based agent is administered no more than about 2 hours before or about 4 hours after administering a radiographic contrast agent. Multiple doses of NAC (e.g., one, two, three or four or more doses) may be administered.

The methods and compositions of the invention are particularly useful for reducing renal toxicity of patients undergoing procedures employing radiographic contrast agents, such as CT scans, angiography procedures or an angioplasty procedures. The methods will also be of particular importance when such procedures are performed on patients having reduced renal function.

In another aspect of the invention, there is provided a pharmaceutical formulation for intravenous administration in a method for reducing organ toxicity associated with a medical diagnostic or therapeutic procedure which employs a radiographic contrast agent, said formulation comprising a thiol-based agent, such as NAC, at a dose in the range of about 600 mg/kg-1500 mg/kg. In more particular embodiments, the formulations comprise NAC at a dose in the range of about 600-1200 or about 800-1200 or about 800-1500 mg/kg.

According to another aspect of the invention, there is provided a method for locally administering a thiol-based compound or agent to an organ or tissue area to protect against injury from diagnostic or therapeutic intra-arterial procedures comprising positioning an intra-arterial catheter in an artery providing blood flow to a potential site or organ of injury and administering, via the positioned arterial catheter, a thiol-based agent. The method is particularly well suited to procedures in which injury is caused by injecting radiographic contrast agent to position an intra-arterial catheter. The thiol-based agent may include, but is not limited to, a compound selected from the group consisting of N-acetyl cysteine (NAC), sodium thiosulfate (STS), GSH ethyl ester, D-methionine, ethyol, and combinations thereof. The thiol-based agent is, in certain embodiments, administered by a catheterization procedure via a catheter having a tip that is located intra-arterially in a position of the circulatory system. The dose of the thiol-based agent per procedure may be in the range of about 20-40 g/m² (equivalent to 0.5 mg/kg-1200 mg/kg in humans). When NAC is used, the dose of NAC agent per procedure will typically be in the range of about 50 mg/kg-1200 mg/kg. In more particular embodiments, the NAC dose is in the range of about 50-200 or 100-400 mg/kg. When STS is used, its dose will typically be in the range of 5 g/m² to 20 g/m².

In another aspect of the invention, a method is provided for reducing nephrotoxicity associated with diagnostic or therapeutic intra-arterial procedures which employ radiographic contrast agents comprising positioning an intra-arterial catheter in an artery providing blood flow to the kidneys and administering, via the positioned arterial catheter, a thiol-based agent. In one embodiment, the thiol-based agent is a compound selected from the group consisting of N-acetyl cysteine (NAC), sodium thiosulfate (STS), GSH ethyl ester, D-methionine, ethyol, and combinations thereof. The intra-arterial procedure may be any procedure in which contrast agents are employed including, but not limited to, cardiac, cerebral or endovascular procedures. In one embodiment, the intra-arterial procedure is an angiography or angioplasty procedure. In another embodiment, the procedure is performed on a patient having reduced renal function. For example, diabetic patients with reduced renal function, in whom coronary angiography is often delayed because of the considerable risks to renal function associated with contrast agents used in the procedure, may find particular benefit in the methods of the invention. The thiol-based agent is administered via a catheter tip, wherein the tip is located intra-arterially in the descending aorta in a position above and in close proximity to the renal arteries. The method often further comprises administering radiographic contrast agent through the same catheter, generally no more than about 2 hours before or 4 hours after administering a radiographic contrast agent. In one embodiment, the thiol-based agent is NAC and the NAC is administered at a dose in the range of about 50-1200 mg/kg. In more particular embodiments, the NAC dose is in the range of about 50-200 or 100-400 mg/kg.

According to another aspect of the invention, there is provided a method for reducing nephrotoxicity associated with intra-arterial catheterization procedures which employ radiographic contrast agents comprising positioning an intra-arterial catheter in a femoral artery, advancing the catheter into the descending aorta and administering a thiol-based agent through the catheter in a location above and in close proximity to the renal arteries. In one embodiment, the thiol-based agent is a compound selected from the group consisting of N-acetyl cysteine (NAC), sodium thiosulfate (STS), GSH ethyl ester, D-methionine, ethyol, and combinations thereof. The intra-arterial procedure may be any procedure in which contrast agents are employed including, but not limited to, cardiac, cerebral or endovascular procedures. In one embodiment, the intra-arterial procedure is an angiography or angioplasty procedure. In another embodiment, the procedure is performed on a patient having reduced renal function. The method often further comprises administering radiographic contrast agent through the same catheter used to administer thiol-based compound. The thiol-based compound is generally administered no more than about 2 hours before or 4 hours after administering a radiographic contrast agent. In one preferred embodiment, the thiol-based agent is NAC and the NAC is administered at a dose of NAC in the range of about 50-1200 mg/kg. In more particular embodiments, the NAC dose is in the range of about 50-200 or 100-400 mg/kg.

The methods and compounds will best be understood by reference to the following detailed description of the preferred embodiment, taken in conjunction with the accompanying drawings. The discussion below is descriptive, illustrative and exemplary and is not to be taken as limiting the scope defined by any appended claims.

### BRIEF DESCRIPTION OF THE DRAWINGS

FIG. 1 shows an anatomical, diagram of major arteries and the top level for placing the catheter for administration of the thiol-based protectant agent.

FIG. 2 shows Western Blots demonstrating the protective effect of NAC on cellular apoptosis.

FIGS. 3-4 shows the effects of route of administration of NAC on nephroprotection in rat models of nephrotoxicity.

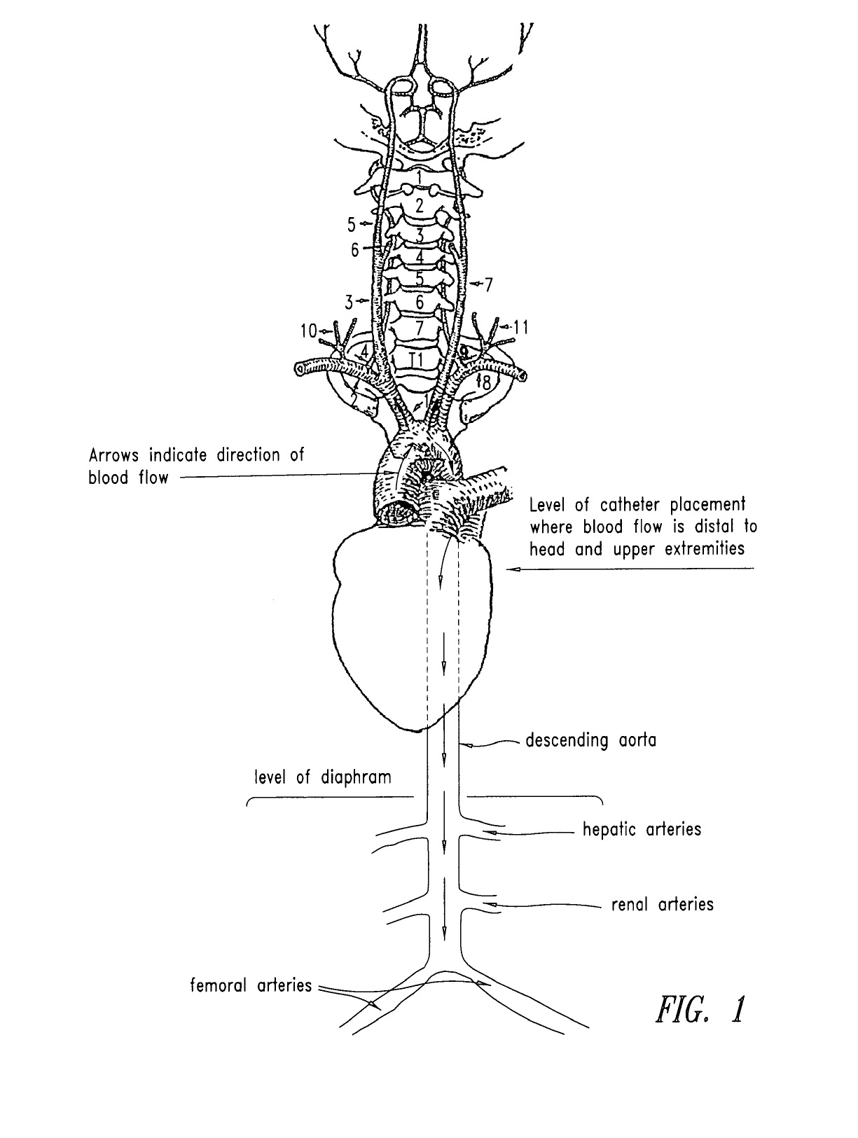

### DETAILED DESCRIPTION OF THE INVENTION

As noted above, the present invention is drawn generally to improved methods for reducing organ toxicity associated with medical procedures which employ radiographic contrast agents. Such agents are well known to cause nephrotoxicity and other complications. The invention is based in part on the identification of improved dosing, timing and delivery strategies for the administration of thiol-based agents and, as further described herein, particular dosing and delivery strategies will be preferred depending on the particular agent used and procedure being performed. A thiol-based agent is a thiol-containing compound effective for reducing nephrotoxicity caused by radiographic contrast agents. These may include, but are not limited to, a compound selected from the group consisting of N-acetyl cysteine (NAC), sodium thiosulfate (STS), GSH ethyl ester, D-methionine, Ethyol, and combinations thereof.

The invention, in certain aspects, provides methods for high-dose intravenous delivery of thiol-based agents, such as NAC, in order to reduce or eliminate nephrotoxicity associated with contrast agents. These methods are useful in the context of any medical imaging procedure which employs radiographic contrast agents including, but not limited to, cardiac, cerebral and endovascular procedures. In certain embodiments, the medical imaging procedure is one that does not require intra-arterial catheterization, including CT scans and the like. The present invention has found, surprisingly, that high-dose intravenous administration of thiol-based agent is a well tolerated and efficacious delivery route for reducing nephrotoxicity. For high-dose intravenous delivery of thiol-based agent, preferred doses will typically be in the range of about 600 mg/kg-1500 mg/kg. In more particular embodiments, NAC is administered intravenously at a dose in the range of about 600-1200 or about 800-1200 or about 800-1500 mg/kg.

Certain other embodiments of the invention use intra-arterial administration and are based on a spatial two-compartment pharmacokinetic model that results in a general tissue first pass effect to prevent significant thiol-based agent from gaining general systemic circulation through the venous circulatory system. Thus, there is a need for only one pass going to tissues of the renal system. This prevents decreased renal function through regionalization of doses of the radiographic agent to the area where radiography is to be performed and doses the thiol-based agent to the renal system.

Therefore, in certain embodiments, the thiol-based agent is administered intra-arterially, in the context of a cardiac, cerebral or endovascular procedure. For example, a thiol-based agent may be administered intra-arterially in a location that provides blood flow to an organ of interest. In a particular embodiment, the thiol-based agent is administered intra-arterially in a location that provides blood flow to the kidneys, for example in the descending aorta, and preferably above and in close proximity to the renal arteries. In this way, the highest concentration of thiol-based agent is provided to the location of organ damage to protect against reduced renal function caused by contrast agents. In another particular embodiment, the thiol-based agent is administered through a catheter located in the femoral artery for a cardiac, cerebral or endovascular procedure, and thiol-based agent is administered as the catheter used for the procedure is advanced above the renal artery.

For intra-arterial procedures in which the thiol-based agent is administered through an intra-arterial catheter having a tip located above the renal arteries, illustrative doses may be in the range of about 50-1500 or about 50-1200 mg/kg, and preferably in the range of about 50-200 or about 100-400 mg/kg. In a particular embodiment, after placing a femoral catheter for a cardiac, cerebral or endovascular procedure, a dose of NAC in the range of about 100-400 mg/kg is administered as the catheter is advanced above and in close proximity to the renal arteries.

An effective dose of the thiol-based protective agent may be administered at any suitable time relative to administration of a contrast agent, provided that nephrotoxicity is reduced or prevented as described herein. In one embodiment, an effective dose of the thiol-based protective agent, such as NAC, is administered no more than about 4 hours prior to administration of contrast agent, and within about 8 hours after administration of the contrast agent. In another embodiment, the thiol-based agent is administered no more than about 2 hours before or about 4 hours after administering a radiographic contrast agent. In addition, multiple doses (e.g., one, two, three or four doses) may be administered as suitable or necessary for a given procedure.

The skilled artisan will understand that any of a number of administration timing, dosing and delivery strategies may be employed while still achieving the objectives of the invention. Accordingly, all such schedules are considered within the spirit and scope of the invention.

Radiographic contrast agent may be administered by the same catheter that is used for administration of thiol-based agent. Preferably, for intra-arterial administration, the thiol-based agent is administered via the arterial catheter one or a plurality of times during the procedure.

The catheter for delivery of the thiol-based agent may be inserted into the arterial system at any suitable site. In one embodiment, the thiol-based agent is administered by a catheterization procedure via a catheter having a tip that is located in the arterial system downstream of the aorta and directed in the mesenteric artery system. In another embodiment, the thiol-based agent is administered by a catheterization procedure via a catheter having a tip that is located in the descending aorta. In one preferred embodiment, the catheter is inserted into a femoral artery and thiol-based agent is administered via a catheter having a tip that is located in the descending aorta, above and in close proximity to the renal arteries, in order to provide the most effective and localized delivery to the kidneys.

### Pharmaceutical Formulations

The present invention further provides pharmaceutical compositions for use in methods for protecting against injury from diagnostic or therapeutic intra-arterial procedures. In one embodiment, the pharmaceutical composition is for use in an intravenous procedure and comprises a thiol-based agent and a contrast agent, wherein the concentration of the thiol-based agent is as discussed above. In another embodiment, the pharmaceutical composition is for use in an intra-arterial procedure and comprises a thiol-based agent and a contrast agent, wherein the concentration of the thiol-based agent is as discussed above.

The compositions and compounds of the present invention may be made and used in essentially any known manner, e.g., by means of conventional mixing, dissolving, emulsifying, encapsulating, entrapping, or lyophilizing processes. Pharmaceutical compositions for use in accordance with the present invention thus may be formulated in any conventional manner using one or more physiologically acceptable carriers comprising excipients and/or auxiliaries that facilitate processing of the active compounds into preparations, which can be used pharmaceutically. Proper formulation is, of course, dependent upon the route of administration chosen.

Techniques for the formulation and administration of the compositions of the present invention are well known, examples of which can be found described in “Remington's Pharmaceutical Sciences” Mack Publishing Co., Easton, Pa., latest edition.

Certain compositions of the invention will comprise a pyrogen-free, sterile solution optionally containing one or more of a reducing agent, a buffer to maintain pH at or near physiologic pH and/or a metal chelating agent to bind metal ions that can catalyze oxidation of the thiol-based agent. For example, the reducing agent may be selected from the group consisting of vitamin E, tocopherol, dithiothreitol, mercaptoethanol, glutathione, and combinations thereof. The buffer will generally be one that is relatively non-toxic and can maintain a pH of between 6 and 8 (e.g., phosphate buffer, Tris buffer). The thiol-based agent may also be stored in a vial having a blanket of an inert gas, e.g., argon, helium, nitrogen and mixtures thereof.

For injection, the compounds of the invention may be formulated in aqueous solutions, preferably in physiologically compatible buffers, such as Hank's solution, Ringer's solution, or physiological saline buffer. The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulary agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Alternatively, the active ingredient may be in powder form for constitution with a suitable vehicle, e.g., sterile pyrogen-free water, before use. In one embodiment, a reducing agent or an anti-oxidant agent is added to the formulation of the thiol-based protective agent to prevent oxidation of the thiol-based protective agent. The antioxidant may include, but is not limited to, vitamin E, tocopherol, dithiotreitol, mercaptoethanol, glutathione. In one embodiment, an inert or non-oxidizing gas is added to a vial for intra-arterial administration. Examples of such gasses are nitrogen, argon, helium, and combinations/mixtures thereof.

A therapeutically effective dose refers to that amount of the compound that results in a reduction in the development or severity of nephrotoxicity associated with radiographic contrast agents. Toxicity and therapeutic efficacy of such compounds can be determined by standard pharmaceutical, pharmacological, and toxicological procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Compounds that exhibit high therapeutic indices are preferred. The data obtained from cell culture assays or animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage will, of course, be chosen by the individual physician in view of the subject being treated, the subject's weight, the severity of the affliction, etc. (See e.g. Fingl et al., 1975, in “The Pharmacological Basis of Therapeutics”, Ch. 1).

### EXAMPLES

### Example 1

### N-Acetylcysteine Safety and Pharmacokinetics

N-acetylcysteine Dose Escalation

Rats were given NAC IV at time 0, 4 hours, and 8 hours and were then followed for two weeks. At 1500 mg/kg×3, animals tolerated the infusions well but lost 5%-10% of body weight within 2-3 days of infusion. At 1200 mg/kg×3, rats showed no weight loss and no signs of toxicity. Thus, three IV administrations of 1200 mg/kg NAC, 4 hours apart, were determined to be safe and well-tolerated.

Effect of Route of Administration on N-Acetylcysteine Biodistribution

Radiolabelled NAC in combination with unlabeled NAC (140 mg/kg) was administered to rats with the following routes of infusion: IV; IA into the right carotid artery for brain infusion; aortic infusion (IA infusion via the left external carotid artery with left internal artery occlusion) to avoid brain infusion; and IA (right carotid) with osmotic Blood Brain Barrier Disruption (BBBD) to maximize delivery to the brain. When NAC was administered IV, negligible amounts were found in the brain. Intra-arterial delivery in the right carotid artery resulted in high levels of radiolabel in the right cerebral hemisphere, which was doubled by BBBD. “Aortic infusion” administered to prevent infusion of the brain, reduced brain delivery of NAC to negligible levels and increased systemic delivery to kidney. These results demonstrate that both IV administration and infusion in the descending aorta gave systemic delivery of NAC without brain delivery.

Effect of Route of Administration and Dose on N-Acetylcysteine Pharmacology

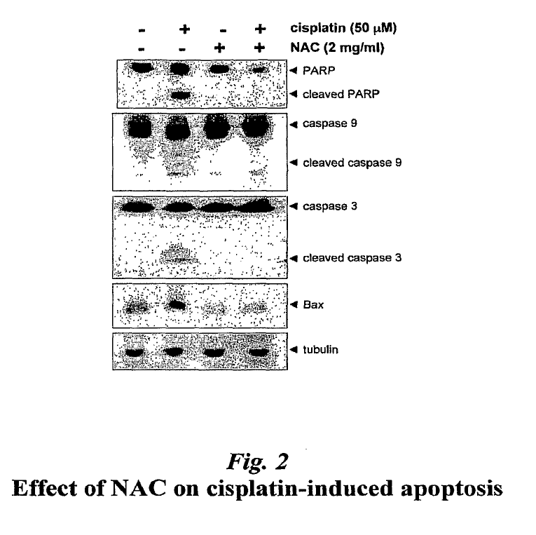

Blood levels of NAC showed a rough linear dose response after IV administration of NAC at 100 (mean=0.38±0.16 mM), 400 (2.19±0.19) and 1200 (6.22±0.18) mg/kg (FIG. 2). The levels of the metabolite oxydized NAC also showed a clear dose response relationship, and reached approximately double the serum concentration as NAC alone at 15 min after administration. HPLC analysis of the IV infusate showed that all the administered NAC was in the non-oxidized form, so all the oxidized NAC in the serum samples was due to metabolic inactivation. NAC is known to enhance de novo glutathione synthesis, and may also impact levels of other thiols. In this HPLC study, glutathione was inconsistently elevated after high dose IV NAC, and there were no change in the levels of the other thiols cysteine or glutathione (GSSG). In contrast to the IV results, the group given NAC 1200 mg/kg by the PO route had very low levels of serum NAC (0.12±0.12) at 15 min or 60 min after infusion.

Clearance of N-Acetylcysteine

NAC was administered either IV or IA into the descending aorta, and blood samples were assessed for thiol concentration every 15 minutes using a colorimetric kit. The NAC doses tested were 140, 400, 1000, and 1200 mg/kg. At all doses, the clearance half life was less than 15 minutes.

### Example 2

### N-Acetylcysteine-Mediated Inhibition of Cisplatin-Induced Apoptosis

Cisplatin cytotoxicity is associated with cellular apoptosis, as evaluated by nuclear translocation of apoptosis induction factor, expression of the pro-apoptotic Bax protein, cleavage of caspases 3 and 9, and cleavage of PARP (e.g., Wu et al., J Pharmacol Exp Therap 312: 424-431, 2005). Thus, the ability of NAC to modulate cellular apoptosis proteins was evaluated by Western Blot analysis. As shown in FIG. 2, NAC administration reversed the cytotoxic effects of cisplatin if added concurrent with cisplatin or up to 2 hours after cisplatin, however protection was reduced if NAC was delayed more than 2 hours and was minimal by 8 hours after cisplatin administration in this assay.

### Example 3

### Importance of Delivery Route and Concentration for N-Acetylcysteine-Mediated Protection Against Nephrotoxicity

There are few animal models of contrast-induced nephropathy, and the reported models are not easily reproducible. Cisplatin chemotherapy induces kidney toxicity in rats as demonstrated by elevated BUN and creatinine, as well as weight loss. In this example, we used cisplatin-induced cytotoxicity and nephrotoxicity, a dose limiting toxicity of cisplatin chemotherapy, as a model of contrast-induced nephropathy.

Rats were treated with cisplatin 10 mg/kg intraperitoneally (IP). NAC at 50 or 400 mg/kg was adminstered to the rats by IP, oral (per os PO) and IV routes and compared to those that received only cisplatin. Rats were tested for renal toxicity 3 days after treatment by measuring serum concentrations of BUN and creatinine (CR).

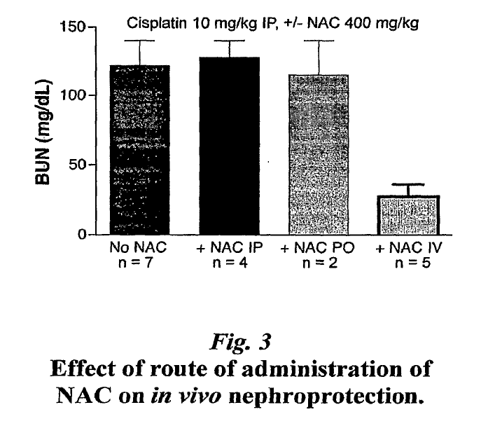

All but one of the rats receiving cisplatin alone had an abnormally high BUN (mean=122±18.0 mg/dL). High level of BUN correlates with renal tubule damage seen histologically. Creatinine values were abnormally high in these animals as well (2.8±0.5). NAC protected against the cisplatin nephrotoxicity but only for certain routes of administration. The rats receiving NAC 400 mg/kg NAC by the IP and PO routes had consistent renal toxicity, as seen in their high BUN (131.8±8.2 and 123.3±8.2, respectively) and CR (2.3±0.38 and 1.77±0.21, respectively) values. When this same dose of NAC was administered by the IV route, however, the rats were protected against cisplatin-induced nephrotoxicity, as shown by the significantly lower BUN (26.33±6.76; p<0.0001) and CR (0.47±0.15; p<0.0013) values. These results are further supported by analysis of weight loss in the different treatment groups. Cisplatin alone caused significant weight loss (approximately 20% loss from basal weight in 3 days). Rats receiving IV NAC had a significantly lower percent weight loss over the test period than the other groups (p<0.0001). Representative results from these experiments are shown in FIG. 3.

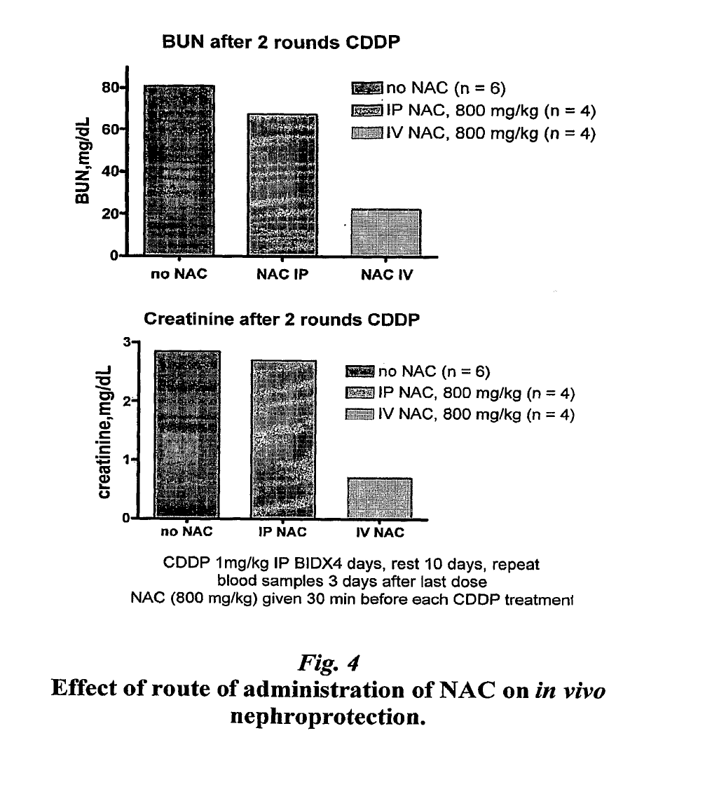

In another study, multiple low dose cisplatin treatments were administered. Cisplatin was administered to rats at 1 mg/kg IP BID, for 4 days, followed by 10 days without treatment. This cisplatin regimen was then repeated. The rats also received either saline or NAC 800 mg/kg IP or IV 30 minutes before each cisplatin treatment. On the third day after the last cisplatin treatment, blood samples were taken, BUN analysis was performed, and creatinine levels were determined. Representative results shown in FIG. 4 demonstrate that there was significant nephrotoxicity caused by this schedule of cisplatin infusion, but that high-dose NAC administered IV was protective against the cumulative nephrotoxicity. NAC administered IP provided no protective effect.

In another study, rats were given a nephrotoxic dose of cisplatin (10 mg/m²) after either NAC IV or PO via a nasogastric tube. NAC via the PO route was not protective against cisplatin nephrotoxicity at 400 mg/kg and these rats had renal failure within 3 days. IV NAC was nephroprotective at a dose of 400 mg/kg but not at a lower dose of 50 mg/kg. Further, although 50 mg/kg NAC was not nephroprotective when administered intravenously, this dose provided significant nephroprotection when administered IA in the descending aorta.

In summary, high doses of NAC administered intravenously were well tolerated and nephroprotective in vivo, while the same doses administered via the IP or PO routes provided minimal protection. This may be due to the NAC being metabolized by first pass through the liver, which occurs more rapidly when it is given IP or PO and thus taken directly into the hepatic portal system. In addition, NAC administered IA at low doses provided significant nephroprotection, equivalent to IV NAC at much higher doses. Therefore, high doses of NAC are well tolerated and necessary for nephroprotection when administering intravenously, whereas much lower doses of NAC can be used when administering intra-arterially.

As noted, the discussion above is descriptive, illustrative and exemplary and is not to be taken as limiting the scope defined by any appended claims.

We claim:

1. A method for reducing organ toxicity associated with a medical diagnostic or therapeutic procedure which employs a radiographic contrast agent comprising administering at least one dose of N-acetyl cysteine (NAC) intravenously, wherein the dose in the range of 600 mg/kg-1500 mg/kg.

2. The method of claim 1, wherein the NAC is administered intravenously at a dose in the range of 600-1200 mg/kg.

3. The method of claim 1, wherein the NAC is administered intravenously at a dose in the range of 800-1200 mg/kg.

4. The method of claim 1, wherein administering the NAC is no more than about 2 hours before administering a radiographic contrast agent.

5. The method of claim 1, wherein administering the NAC is no more than about 4 hours after administering a radiographic contrast agent.

6. The method of claim 1, wherein at least two doses are administered.

7. The method of claim 1, wherein the organ toxicity is nephrotoxicity.

8. The method of claim 1, wherein the procedure is a CT scan, an angiography procedure, or an angioplasty procedure.

9. The method of claim 1, wherein the procedure is performed on a patient having reduced renal function.

10. A pharmaceutical formulation for intravenous administration in a method for reducing organ toxicity associated with a medical diagnostic or therapeutic procedure which employs a radiographic contrast agent, said formulation comprising N-acetyl cysteine (NAC) at a dose in the range of 600-1500 mg/kg.

11. The pharmaceutical formulation of claim 10, wherein the formulation comprises NAC at a dose in the range of 600-1200 mg/kg.

12. The pharmaceutical formulation of claim 10, wherein the formulation comprises NAC at a dose in the range of 800-1200 mg/kg.

13. A method for reducing nephrotoxicity associated with diagnostic or therapeutic intra-arterial procedures which employ radiographic contrast agents comprising positioning an intra-arterial catheter in an artery providing blood flow to the kidneys and administering, via the positioned arterial catheter, a thiol-based agent.

14. The method of claim 13, wherein the thiol-based agent is a compound selected from the group consisting of N-acetyl cysteine (NAC), sodium thiosulfate (STS), GSH ethyl ester, D-methionine, ethyol, and combinations thereof.

15. The method of claim 13, wherein the thiol-based agent is NAC.

16. The method of claim 15, wherein the NAC is administered through the catheter at a dose in the range of 100-400 mg/kg.

17. The method of claim 13, wherein the intra-arterial procedure is a cardiac, cerebral or endovascular procedure.

18. The method of claim 13, wherein the intra-arterial procedure is an angiography or angioplasty procedure.

19. The method of claim 13, wherein procedure is performed on a patient having reduced renal function.

20. The method of claim 13, wherein the thiol-based agent is administered via the catheter tip, wherein the tip is located intra-arterially in the descending aorta in a position above the renal arteries.

21. The method of claim 13, further comprising administering radiographic contrast agent through the catheter.

22. The method of claim 13, wherein administering the thiol-based agent is no more than about 2 hours before administering a radiographic contrast agent.

23. The method of claim 13 wherein administering the thiol-based agent is no more than about 4 hours after administering a radiographic contrast agent.

24. A method for reducing nephrotoxicity associated with intra-arterial catheterization procedures which employ radiographic contrast agents comprising positioning an intra-arterial catheter in a femoral artery, advancing the catheter into the descending aorta and administering a thiol-based agent through the catheter in a location above the renal arteries.

25. The method of claim 24, wherein the thiol-based agent is a compound selected from the group consisting of N-acetyl cysteine (NAC), sodium thiosulfate (STS), GSH ethyl ester, D-methionine, ethyol, and combinations thereof.

26. The method of claim 24, wherein the thiol-based agent is NAC.

27. The method of claim 26 wherein the NAC is administered through the catheter at a dose in the range of 100-400 mg/kg.

28. The method of claim 24, wherein the intra-arterial procedure is an angiography or angioplasty procedure.

29. The method of claim 24, wherein the procedure is performed on a patient having reduced renal function.

30. The method of claim 24, further comprising administering radiographic contrast agent through the catheter.

31. The method of claim 24, wherein administering the thiol-based agent is no more than about 2 hours before administering a radiographic contrast agent.

32. The method of claim 24, wherein administering the thiol-based agent is no more than about 4 hours after administering a radiographic contrast agent.
